(19) United States
(12) Patent Application Publication
(10) Pub. No.: US 2023/0397843 A1
(43) Pub. Date: Dec. 14, 2023

(54) INFORMATIVE DISPLAY FOR NON-CONTACT PATIENT MONITORING

(71) Applicant: Covidien LP, Mansfield, MA (US)

(72) Inventors: Paul S. ADDISON, Edinburgh (GB); Dean MONTGOMERY, Edinburgh (GB); Philip C. SMIT, Hamilton (GB)

(21) Appl. No.: 18/330,925

(22) Filed: Jun. 7, 2023

Related U.S. Application Data
(60) Provisional application No. 63/366,158, filed on Jun. 10, 2022.

(51) Int. Cl. A61B 5/113; A61B 5/00

(52) U.S. Cl. CPC A61B 5/113; A61B 5/743

(57) ABSTRACT

The disclosed technology provides image-based patient monitoring by capturing a first image, by an image capture sensor, of a predefined region of a patient at a first time, capturing a second image, by the image capture sensor, of the predefined region of the patient at a second time, determining whether the first captured image and the second captured image satisfy a non-respiratory motion condition, the non-respiratory motion condition based in part on a predefined relationship between captured images and motion of a patient attributable to patient actions other than respiration of the patient, classifying a motion of the patient as non-respiratory motion when the non-respiratory motion condition is satisfied, and transmitting an instruction to display an indication of veracity of a respiratory measurement of the patient measured between the first time and the second time based on the classified motion.

### CROSS-REFERENCE TO RELATED APPLICATIONS

This application claims priority to U.S. Provisional Patent Application No. 63/366,158, filed Jun. 10, 2022, and entitled INFORMATIVE DISPLAY FOR NON-CONTACT PATIENT MONITORING, the entirety of which is hereby incorporated by reference.

### TECHNICAL FIELD

The present disclosure relates to informative displays for non-contact patient monitoring, and more specifically, to informative displays for visualizing information that may affect the veracity of respiratory rate and/or other physiological measures. The information may be calculated from depth measurements taken and/or images captured by a non-contact patient monitoring system, including a depth-sensing camera, and the information informing the veracity of respiratory measurements may be derived from the information. The information may include classifications representing gross non-respiratory patient motions. The information may be configured or otherwise formatted for display in conjunction with the respiratory rate and/or other physiological measures.

### BACKGROUND

Depth sensing technologies have been developed that, when integrated into non-contact patient monitoring systems, can be used to determine a number of physiological and contextual parameters, such as respiration rate, tidal volume, minute volume, etc. Such parameters can be displayed on a display so that a clinician is provided with a basic visualization of these parameters. For example, respiratory rate measurements may be presented.

However, additional effort and analysis may be required for the clinician to decipher and interpret what the displayed data means with respect to the health of the patient being monitored. For example, the respiratory rate measurements can be impacted by gross movements of a patient that are not attributable to patient respiration. Accordingly, a need exists for systems and methods that are capable of both synthesizing patient monitoring data and providing additional visualization to indicate the veracity of the respiratory rate measurements when non-respiratory patient motion is detected.

### SUMMARY

In some embodiments, the disclosed technology provides image-based patient monitoring by capturing a first image, by an image capture sensor, of a region of a patient at a first time, capturing a second image, by the image capture sensor, of the region of the patient at a second time, determining whether the first captured image and the second captured image satisfy a non-respiratory motion condition, the non-respiratory motion condition based in part on a relationship between captured images and motion of a patient attributable to patient actions other than respiration of the patient, classifying a motion of the patient as non-respiratory motion when the non-respiratory motion condition is satisfied, and transmitting an instruction to display a visual indicator on a graph plotting the patient's respiration measurements taken over time, the visual indicator providing an indication of whether the motion of the patient has been classified as non-respiratory motion.

In some embodiments, the disclosed technology provides an image-based patient monitoring system. The system includes one or more hardware processors operable to execute instructions stored in memory and a distance sensor operable to generate a first distance signal based on a detected at least one first distance between at least one point in a region of a patient and the distance sensor at a first time and generate a second distance signal based on a detected at least one second distance from the at least one point in the region of the patient and the distance sensor at a second time. The system further includes a signal processor executable by the one or more hardware processors and operable to determine whether the first generated distance signal and the second generated distance signal satisfy a non-respiratory motion condition, the non-respiratory motion condition based in part on a relationship between generated distance signals and motion of a patient attributable to patient actions other than respiration of the patient and classify a motion of the patient as non-respiratory motion when the non-respiratory motion condition is satisfied. The system further includes an instruction generator executable by the one or more hardware processors and operable to generate an instruction to display a visual indicator on a graph plotting the patient's respiration measurements taken over time, the visual indicator providing an indication of whether the motion of the patient has been classified as non-respiratory motion.

In some embodiments, the disclosed technology provides image-based patient monitoring by generating a first distance signal based on at least one first distance between at least one point in a region of a patient and a distance sensor of an image capture sensor at a first time, generating a second distance signal based on at least one second distance from the at least one point in the region of the patient and the distance sensor at a second time, determining whether the first generated distance signal and the second generated distance signal satisfy a non-respiratory motion condition, the non-respiratory motion condition based in part on a relationship between generated distance signals and motion of a patient attributable to patient actions other than respiration of the patient, classifying a motion of the patient as non-respiratory motion when the non-respiratory motion condition is satisfied, and transmitting an instruction to display an indication of veracity of a respiratory measurement of the patient measured between the first time and the second time based on the classified motion.

In some embodiments, the disclosed technology provides an image-based patient monitoring system. The system includes one or more hardware processors operable to execute instructions stored in memory and an image capture sensor operable to capture a first image of a region of a patient at a first time and capture a second image of the region of the patient at a second time. The system further includes a signal processor executable by the one or more hardware processors and operable to determine whether the first captured image and the second captured image satisfy a non-respiratory motion condition, the non-respiratory motion condition based in part on a relationship between captured images and motion of a patient attributable to patient actions other than respiration of the patient and to classify a motion of the patient as non-respiratory motion when the non-respiratory motion condition is satisfied. The system further includes an instruction generator executable by the one or more hardware processors and operable to generate an instruction to display an indication of veracity of a respiratory measurement between the first time and the second time based on the classified motion.

### BRIEF DESCRIPTION OF THE DRAWINGS

Many aspects of the present disclosure can be better understood with reference to the following drawings. The components in the drawing are not necessarily to scale. Instead, emphasis is placed on illustrating clearly the principles of the present disclosure. The drawings should not be taken to limit the disclosure to the specific embodiments depicted but are for explanation and understanding only.

FIG. 1 is a schematic view of an implementation of a video-based patient monitoring system configured in accordance with various embodiments of the present technology.

FIG. 2 is a block diagram illustrating an implementation of a video-based patient monitoring system having a computing device, a server, and one or more image capturing devices, and configured in accordance with various embodiments of the present technology.

FIG. 3 is a plot view of line plots illustrating signals from a transthoracic impedance respiratory measurement as a function of time.

FIG. 4 is a display view for an implementation of a user interface of a video-based patient monitoring system configured in accordance with various embodiments of the present technology.

FIG. 5 is a flow chart of an implementation of a method for providing an informative display of data obtained from non-contact monitoring of a patient configured in accordance with various embodiments of the present technology.

### DETAILED DESCRIPTION

The present disclosure relates to informative displays for non-contact patient monitoring. The technology described herein can be incorporated into systems and methods for non-contact patient monitoring. As described in greater detail below, the described technology can include obtaining respiratory data, such as via non-contact patient monitoring using image sensors (e.g., depth-sensing cameras), and displaying the respiratory data. The technology may further include comparing captured image data of a portion of a patient and/or generated distance data relative to a portion of a patient to determine whether a patient's motion is attributable to breathing or respiration or is attributable to patient actions other than respiration and classify the patient's motion accordingly. When patient motion is classified as attributable to non-respiratory patient motion, the motion may indicate that traditional respiratory measurements such as respiratory rate have less veracity than in times when motion is largely attributable to respiration. Specifically, gross movements of a patient attributable to sources other than respiration can introduce motion signals of a larger magnitude that obfuscate the smaller motions and the substantially oscillatory signal behavior attributable to respiration. The technology may provide an instruction to display an indication of the veracity of a respiratory measurement based on the determination and classification.

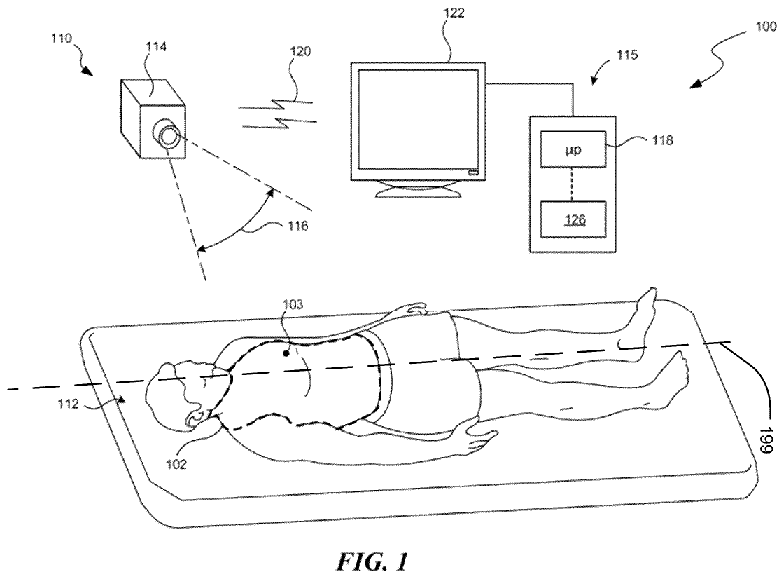

Specific details of several embodiments of the present technology are described herein with reference to FIGS. 1-5. Although many of the embodiments are described with respect to devices, systems, and methods for image-based monitoring of breathing in a human patient and associated display of this monitoring, other applications and other embodiments in addition to those described herein are within the scope of the present technology. For example, at least some embodiments of the present technology can be useful for image-based monitoring of breathing in other animals and/or in non-patients (e.g., elderly or neonatal individuals within their homes). It should be noted that other embodiments, in addition to those disclosed herein, are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

FIG. 1 is a schematic view of a patient 112 and an implementation of a video-based patient monitoring system 100 configured in accordance with various embodiments of the present technology. The system 100 includes a non-contact detector 110 and a computing device 115. In some embodiments, the non-contact detector 110 can include one or more image capture devices, such as one or more video cameras. In the illustrated embodiment, the non-contact detector 110 includes the camera 114 (which may be a video camera adapted to capture video or other time series image data). The non-contact detector 110 of the system 100 is placed remote from the patient 112. More specifically, the camera 114 of the non-contact detector 110 is positioned remotely from the patient 112 in that it is spaced apart from and does not contact the patient 112. The camera 114 includes a detector exposed to a field of view (FOV) 116 that encompasses at least a portion of the patient 112. In implementations, the camera 114 is operable to detect electromagnetic energy of any spectrum or other energy (e.g., infrared, visible light, thermal, x-ray, microwave, radio, gamma-ray, and the like).

The camera 114 can capture a sequence of images over time. The camera 114 can be a depth-sensing camera, such as a Kinect camera from Microsoft Corp. (Redmond, Washington) or Intel camera, such as the D415, D435, and SR305 cameras from Intel Corp. (Santa Clara, California). A depth-sensing camera can detect a distance between the camera and objects within its field of view. Such information can be used to determine that a patient 112 is within the FOV 116 of the camera 114 and/or to determine one or more regions of interest (ROI) to monitor on the patient 112. A medial ROI axis 199 is presented. The medial ROI axis 199 is an axis about which the field of view is substantially symmetrical when a patient is not in motion and is lying flat. Once an ROI is identified, the ROI can be monitored over time, and the changes in depth of regions and/or captured images within the ROI 102 can represent movements of the patient 112 associated with breathing.

As described in greater detail in U.S. Patent Application Publication No. 2019/0209046, those movements, or changes of regions within the ROI 102, can additionally or alternatively be used to determine various breathing parameters, such as tidal volume, minute volume, respiratory rate, respiratory volume, etc. Those movements, or changes of regions within the ROI 102, can also be used to detect various breathing abnormalities, as discussed in greater detail in U.S. Patent Application Publication No. 2020/0046302. The various breathing abnormalities can include, for example, low flow, apnea, rapid breathing (tachypnea), slow breathing, intermittent or irregular breathing, shallow breathing, obstructed and/or impaired breathing, and others. U.S. Patent Application Publication Nos. 2019/0209046 and 2020/0046302 are incorporated herein by reference in their entirety.

In some embodiments, the system 100 determines a skeleton-like outline of the patient 112 to identify a point or points from which to extrapolate an ROI. For example, a skeleton-like outline can be used to find a center point of a chest, shoulder points, waist points, and/or any other points on the body of the patient 112. These points can be used to determine one or more ROIs. For example, an ROI 102 can be defined by filling in the area around a point 103 such as a center point of the chest, as shown in FIG. 1. Certain determined points can define an outer edge of the ROI 102, such as shoulder points. In other embodiments, instead of using a skeleton, other points are used to establish an ROI. For example, a face can be recognized, and a chest area inferred in proportion and spatial relation to the face. In other embodiments, a reference point of a patient's chest can be obtained (e.g., through a previous 3-D scan of the patient), and the reference point can be registered with a current 3-D scan of the patient. In these and other embodiments, the system 100 can define an ROI around a point using parts of the patient 112 that are within a range of depths from the camera 114. In other words, once the system 100 determines a point from which to extrapolate an ROI, the system 100 can utilize depth information from the camera 114 (which may be a depth-sensing camera) to fill out the ROI. For example, if the point 103 on the chest is selected, parts of the patient 112 around the point 103 that are a similar depth or distance from the camera 114 as the point 103 are used to determine the ROI 102.

In another example, the patient 112 can wear specially configured clothing (not shown) responsive to visible light or clothing responsive to electromagnetic energy of a different spectrum that includes one or more features to indicate points on the body of the patient 112, such as the patient's shoulders and/or the center of the patient's chest. The one or more features can include a visually encoded message (e.g., bar code, QR code, etc.), and/or brightly colored shapes that contrast with the rest of the patient's clothing. In these and other embodiments, the one or more features can include one or more sensors that are operable to indicate their positions by transmitting light or other information to the camera 114. In these and still other embodiments, the one or more features can include a grid or another identifiable pattern to aid the system 100 in recognizing the patient 112 and/or the patient's movement. In some embodiments, the one or more features can be stuck on the clothing using a fastening mechanism such as adhesive, a pin, etc. For example, a small sticker can be placed on a patient's shoulders and/or on the center of the patient's chest that can be easily identified within an image captured by the camera 114. The system 100 can recognize the one or more features on the patient's clothing to identify specific points on the body of the patient 112. In turn, the system 100 can use these points to recognize the patient 112 and/or to define an ROI.

In some embodiments, the system 100 can receive user input to identify a starting point for defining an ROI. For example, an image can be reproduced on a display 122 of the system 100, allowing a user of the system 100 to select a patient 112 for monitoring (which can be helpful where multiple objects are within the FOV 116 of the camera 114) and/or allowing the user to select a point on the patient 112 from which an ROI can be determined (such as the point 103 on the chest of the patient 112). In other embodiments, other methods for identifying a patient 112, identifying points on the patient 112, and/or defining one or more ROI's can be used.

In an implementation, an ROI may include a chest of the patient 112. The patient's chest typically moves during respiration. The chest cavity is moved to cause the lungs to inflate. The respiratory motion typically presents as consistent alternating chest expansion and relaxation. Detection of the chest ROI can help determine whether the patient's motion is attributable to the respiration of the patient 112. As disclosed herein, respiratory patient motion includes motion attributable to breathing, including chest expansion and relaxation. It is appreciated that all patient motion is tenuously associated with respiration, as respiration drives most processes in a healthy patient. However, respiratory patient motion, as disclosed herein, is directly attributable to breathing motion. Another potential ROI is a patient's nose, monitoring nasal passages to determine whether the nasal passages open or close responsive to exhaling and inhaling, respectively.

The camera 114 can be used to generate sensor data representing one or more of images of the ROI or distances between points in the ROI and the camera 114 to distinguish patient 112 respiratory motion (at least largely) attributable to respiration from non-respiratory motion attributable to patient 112 actions other than respiration. The sensor data generated by the camera 114, including captured images and/or signals representing distances from points within the ROI and the camera 114, can be sent to the computing device 115 through a wired or wireless connection 120. The computing device 115 can include a hardware processor 118 (e.g., a microprocessor), the display 122, and/or hardware memory 126 for storing software and computer instructions. Sequential image frames of the patients and/or distance signals representing distances from the patient 112 are recorded by the camera 114 and sent to the hardware processor 118 for analysis. The analysis may be conducted by a signal processor executable by the hardware processor 118.

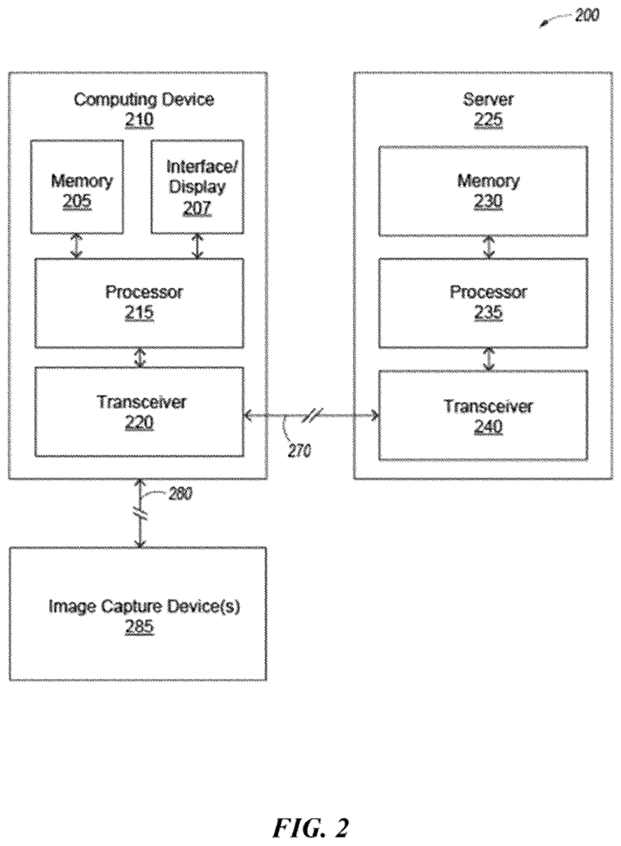

The display 122 can be remote from the camera 114, such as a video screen positioned separately from the hardware processor 118 and the hardware memory 126. Other embodiments of the computing device 115 can have different, fewer, or additional components than shown in FIG. 1. In some embodiments, the computing device 115 can be a server. In other embodiments, the computing device 115 of FIG. 1 can be additionally connected to a server (e.g., as shown in FIG. 2 and discussed in greater detail below). The captured images/video can be processed or analyzed by the signal processor at the computing device 115 and/or a server to determine a variety of parameters (e.g., respiratory patient motion, non-respiratory patient motion, tidal volume, minute volume, respiratory rate, etc.) of the patient's breathing. In some embodiments, some or all of the processing may be performed by the camera, such as by a hardware processor integrated into the camera or when some or all of the computing device 115 is incorporated into the camera.

FIG. 2 is a block diagram illustrating an implementation of a video-based patient monitoring system 200 (e.g., the video-based patient monitoring system 100 shown in FIG. 1) having a computing device 210 (e.g., an implementation of the computing device 115), a server 225, and one or more image capture device(s) 285, and configured in accordance with various embodiments of the present technology. In various embodiments, fewer, additional, and/or different components can be used in the system 200. The computing device 210 includes a hardware processor 215 (e.g., an implementation of the hardware processor 118) that is coupled to a memory 205. The hardware processor 215 can store and recall data and applications in the memory 205, including applications that process information and send commands/signals according to any of the methods disclosed herein. The hardware processor 215 can also (i) display objects, applications, data, etc. on an interface/display 207 and/or (ii) receive inputs through the interface/display 207. As shown, the hardware processor 215 is also coupled to a transceiver 220.

The computing device 210 can communicate with other devices, such as the server 225 and/or the image capture device(s) 285 via (e.g., wired or wireless) connections 270 and/or 280, respectively. For example, the computing device 210 can send to the server 225 information determined about a patient from images captured by the image capture device(s) 285. The computing device 210 can be the computing device 115 of FIG. 1. Accordingly, the computing device 210 can be located remotely from the image capture device(s) 285, or it can be local and close to the image capture device(s) 285 (e.g., in the same room). In various embodiments disclosed herein, the hardware processor 215 of the computing device 210 can perform the steps disclosed herein. In other embodiments, the steps can be performed on a hardware processor 235 of the server 225. The hardware processor 235 of the server 225 is coupled to a memory 230. The hardware processor 235 can store and recall data and applications in the memory 230. The hardware processor 235 is also coupled to a transceiver 240. In some embodiments, the hardware processor 235, and subsequently the server 225, can communicate with other devices, such as the computing device 210, through a connection 270.

In some embodiments, the various steps and methods disclosed herein can be performed by both of the hardware processors 215 and 235. In some embodiments, certain steps can be performed by the hardware processor 215 while others are performed by the hardware processor 235. In some embodiments, information determined by the hardware processor 215 can be sent to the server 225 for storage and/or further processing.

In an implementation, the image capture device(s) 285 generate sensor data such as captured images and/or signals representing distances between the image capture device(s) 285 and at least one point in an ROI. In some embodiments, the image capture device(s) 285 are remote sensing device(s), such as depth-sensing video camera(s), as described above with respect to FIG. 1.

In some embodiments, the image capture device(s) 285 can be or include some other type(s) of device(s), such as proximity sensors or proximity sensor arrays, heat or infrared sensors/cameras, sound/acoustic or radio wave emitters/detectors, or other devices that include a field of view and can be used to monitor the location and/or characteristics of a patient or a region of interest (ROI) on the patient. Body imaging technology can also be utilized according to the methods disclosed herein. For example, backscatter x-ray or millimeter-wave scanning technology can be utilized to scan a patient, which can be used to define and/or monitor an ROI. Advantageously, such technologies can be able to penetrate (e.g., “see”) through clothing, bedding, or other materials while giving an accurate representation of the patient's skin. This can allow for more accurate measurements, particularly if the patient is wearing baggy clothing or is under bedding. The image capture device(s) 285 can be described as local because they are relatively close in proximity to a patient such that at least a part of a patient is within the field of view of the image capture device(s) 285.

In some embodiments, the image capture device(s) 285 can be adjustable to ensure that the patient is captured in the field of view. For example, the image capture device(s) 285 can be physically movable, can have a changeable orientation (such as by rotating or panning), and/or can be capable of changing a focus, zoom, or other capture characteristic to allow the image capture device(s) 285 to adequately capture images of a patient and/or an ROI of the patient. In various embodiments, for example, the image capture device(s) 285 can focus on an ROI, zoom in on the ROI, center the ROI within a field of view by moving the image capture device(s) 285, or otherwise adjust the field of view to allow for better and/or more accurate tracking/measurement of the ROI.

In an implementation, the generated sensor data can include time-series data. For example, the sensor data can be arranged chronologically, perhaps with associated timestamps representing data capture and/or generation times. The time-series data may represent patient motion over time. The time-series data can represent video data for captured images and can represent changes in distances between the image capture device(s) 285 and points in the ROI for distance signal data. The time-series data can be analyzed to show patient motion over time and/or changes in patient motion over time and can be used to distinguish between respiratory patient motion and non-respiratory patient motion. When time-series data is analyzed, durations including one or more time windows (e.g., between a first time and a second time) and/or sample sizes may be used during the analysis. The durations may be dynamically determined based on the breathing patterns of a particular patient or may be standardized.

The system 200 may include a signal processor for processing the sensor data generated by the image capture device(s) 285. The signal processor may include a hardware element of one or more of the computing device 210, the image capture device(s) 285, and the server 225, may include a software element executable by one or more of a processor of the image capture device(s) 285, the hardware processor 215, and the hardware processor 235, or may include a hybrid system of software and hardware contained in one or more of the computing device 210, the image capture device(s) 285, and the server 225.

The signal processor receives the generated sensor data and is operable to determine whether the generated sensor data satisfies a non-respiratory motion condition. The non-respiratory motion condition and/or its satisfaction may be determined based on a relationship accessible to the signal processor between generated sensor data and motion of a patient attributable to patient actions other than respiration. Non-respiratory patient motions attributable to patient actions other than respiration can affect the veracity of respiratory measurements (e.g., respiratory rate) by obfuscating signals associated with respiratory patient motions that are typically smaller in magnitude and show regular oscillations associated with alternating lung expansion and relaxation.

The non-respiratory motion condition may include a range or threshold value of relevant metrics such as changes in distances, asymmetrical changes of distances (e.g., relative to a medial ROI axis), changes in pixel values in captured images, and the like. In an implementation, asymmetric motion detected in an ROI on a patient's chest can indicate the motion is attributable to something other than breathing. In another implementation, a magnitude of motion in the ROI may be indicative of a gross patient movement not associated with patient breathing. In implementations, the non-respiratory motion condition may account for simultaneous gross movement inside and outside of an ROI, which may indicate that the camera is moving (e.g., being bumped or jostled by someone). In implementations, the non-respiratory motion condition accounts for a sum of changes in pixel distances, a sum of absolute values of distances, and/or whether the one more of the sums exceeds a threshold. In implementations, the non-respiratory motion condition may be based on specific pixels (e.g., a localized group) identified as representing or covering all or a part of one or more of a patient, a patient's torso, a patient's hand, and a patient's head.

The non-respiratory condition may be based on a demographic or other quality of the patient. Patient demographics upon which the non-respiratory condition may be based can include one or more of age, size, race, gender, body type, and the like. For example, a specific non-respiratory condition may apply to neonates. Neonates may present very significant differences between respiratory neonate patient motion and non-respiratory neonate patient motion than adults. Also, non-respiratory neonate patient motion may be more uniform among neonates than non-respiratory adult patient motion is among adults. In certain situations, the neonate may be in an isolette or a bassinet, which may alter considerations of neonate breathing and imaging. Neonates in isolettes tend to be very small, largely incapable of turning or rotating, and are typically clothed. Larger neonates and/or older babies may be more likely to turn and are less consistently clothed and may be covered with a blanket and/or swaddled.

The signal processor may include a patient motion classifier operable to classify patient motion based on the satisfaction of one or more non-respiratory motion conditions. For example, a single patient motion classification can be based on satisfaction of more than one non-respiratory condition. Because the classifications represent an extent to which a patient motion is attributable to non-respiratory patient motion, and non-respiratory motion is correlated with poor veracity in respiratory measurements, the classifications of the patient motion may represent the veracity of respiratory measurements (e.g., based on predefined parameters of veracity).

In implementations, the signal processor may be operable to determine a level of lethargy of a patient based on the generated sensor data. For example, in neonates, the baseline activity of the neonate reflected in non-respiratory movements can be an indicator of health of a neonate. The signal processor may determine the lethargy using a predefined relationship between the generated sensor data generated over a first duration relative to the generated sensor data generated over a second duration, or the generated sensor data may be compared with preexisting data associated with lethargy to the satisfaction of a lethargy condition (e.g., a threshold relative motion pattern predefined to indicated lethargy). Levels of lethargy may be elements of predefined relationships between generated sensor data and non-respiratory patient motion and/or may be elements of non-respiratory motion conditions associated with the predefined relationships.

In implementations, the patient motion may be classified in a binary fashion as one of respiratory patient motion or of non-respiratory patient motion (e.g., sufficient gross non-respiratory patient motion to flag measurements made during the time of the motion as lacking a predefined requisite veracity). In other implementations, the patient motion may be classified based on an extent of motion attributable to each of respiratory patient motion and non-respiratory patient and may additionally or alternatively include or be based on a determined confidence that the patient motion is attributable to respiratory motion and/or non-respiratory motion. The motion classifications can be representative of different degrees of the patient motion's attributability to non-respiratory patient motion and/or different degrees of confidence in the determination of the attributability. For example, the motion classifier may generate one or more of a score associated with the determination of the attributability and a score associated with confidence in the determination of the attributability to classify patient motion. In implementations in which a confidence score is determined, the confidence score may be combined with the score associated with the attributability (e.g., multiply or otherwise weigh) to classify patient motion.

In implementations, the patient motion classifier associates each motion classification with a motion classification flag and generates a motion classification flag when the motion classifier classifies an associated patient motion. The motion classification flag may be set to inform elements of the system 200 of a classification of patient motion.

The signal processor can additionally or alternatively determine other respiratory measurements such as values of a variety of parameters (e.g., respiratory patient motion, non-respiratory patient motion, tidal volume, minute volume, respiratory rate, etc.). In implementations, the signal processor can determine the respiratory measurements based on the generated sensor data. In other implementations, the signal processor can receive respiratory measurements generated by other means, including one or more of a transthoracic impedance measurement, an electrocardiogram, capnograph, spirometer, pulse oximeter, and a manual user entry. Implementations are also contemplated in which the signal processor does not process respiratory measurements other than the classification of patient motion. In implementations, the respiratory measurements may inform when the signal processor classifies patient motion. For example, the signal processor may be configured to monitor patient motion and/or generate sensor data responsively to a detection of anomalous respiratory measurements (e.g., when the monitored respiratory measurements satisfy an anomalous respiratory measurement condition based on a predefined threshold or range of values of the respiratory measurements or changes in the respiratory measurements). In implementations, determinations and/or outputs of the signal processor can be sent to systems storing electronic records to add the outputs to the electronic records.

In implementations the system includes a respiratory activity analyzer executable by a hardware processor (e.g., one or more of hardware processor 215, hardware processor 235, or a hardware processor of the image capture device(s) 285) to analyze respiratory activity of the patient based on raw respiratory measurement data. For example, the respiratory activity analyzer may receive a raw respiratory motion signal (e.g., a TTI signal) and process the raw respiratory motion signal to determine a respiratory rate (or another derivative respiratory measurement). In implementations, the respiratory activity analyzer may determine the respiratory rate (or another derivative respiratory measurement) exclusively during periods in which the motion of the patient is classified as attributed substantially to respiration of the patient.

The signal processor and/or an instruction generator executable by a hardware processor (e.g., one or more of hardware processor 215, hardware processor 235, or a hardware processor of the image capture device(s) 285) of the system 200 can generate instructions for the display of data generated by the signal processor. An instruction can include data representing an instruction to display an indication of the veracity of a respiratory measurement generated during a duration of analysis of patient motion based on the classification of the patient motion. The instruction may alternatively or additionally include data representing a respiratory measurement (e.g., one generated by the signal processor). In implementations, the instruction and/or indication may include data representing one or more of a motion classification flag, a classification-specific display, an overlaid display (e.g., configured to overlay a displayed element in a user interface), an image representation of patient motion (e.g., a visual or video representation of captured images and/or generated distance signals), an audio signal, a flashing element (e.g., where the magnitude of light of elements of a display is alternatively increased and decreased), a different alert, a code representing the aforementioned items, and the like. In implementations in which the classification is one of a number of classifications, each classification may correspond to a different display. The signal processor may output data representing the motion classification or a specific display associated in memory of the system 200 with the motion classification. For example, different classifications can be represented in a display by different colors, different magnitudes of light in the display, different frequencies at which to flash light in the display, and the like. In implementations in which the motion classifications are representative of different degrees of the patient motion's attributability to non-respiratory patient motion and/or different degrees of confidence in the determination of the attributability, the corresponding display may represent a spectrum or range of color, light magnitude, or flash frequency based on a magnitude of the one or more of the different degrees of the patient motion's attributability to non-respiratory patient motion and/or different degrees of confidence in the determination of the attributability.

In implementations, the instruction or indication may be configured to cause a display to display the indication of the classification as an element overlaid over or underlaid under (e.g., displayed behind) another displayed respiratory measurement or an image of the patient, including an ROI image to indicate the veracity of the displayed respiratory measurement. For example, the display may be configured to display a measured respiratory rate over time and may display the indication of veracity of the measured respiratory rate over or under the displayed respiratory rate. An overlaid or underlaid display of the indication may be configured to be visually contrasted from the displayed respiratory measurement. For example, the elements may be of different colors and/or may be of different transparencies. For example, the displayed indication of veracity may be at least partially transparent to maintain visibility of the displayed respiratory measurement (e.g., appearing as a highlighting of or patch over the displayed respiratory measurement). Additionally or alternatively, the displayed indication of veracity may be overlaid over an ROI image. In an alternative implementation, the instruction instructs a display to not display the respiratory measurements when patient motion is classified as non-respiratory motion and/or display that the non-respiratory motion is occurring.

The devices shown in the illustrative embodiment can be utilized in various ways. For example, either the connections 270 and 280 can be varied. Either of the connections 270 and 280 can be a hard-wired connection. A hard-wired connection can involve connecting the devices through a USB (universal serial bus) port, serial port, parallel port, or other type of wired connection that can facilitate the transfer of data and information between a processor of a device and a second processor of a second device. In another embodiment, either of the connections 270 and 280 can be a dock where one device can plug into another device. In other embodiments, either of the connections 270 and 280 can be a wireless connection. These connections can take the form of any sort of wireless connection, including, but not limited to, Bluetooth connectivity, Wi-Fi connectivity, infrared, visible light, radio frequency (RF) signals, or other wireless protocols/methods. For example, other possible modes of wireless communication can include near-field communications, such as passive radio-frequency identification (RFID) and active RFID technologies. RFID and similar near-field communications can allow the various devices to communicate over a short range when they are placed proximate to one another. In yet another embodiment, the various devices can connect through an internet (or other network) connection. That is, either of the connections 270 and 280 can represent several different computing devices and network components that allow the various devices to communicate through the internet, either through a hard-wired or wireless connection. Either of the connections 270 and 280 can also be a combination of several modes of connection.

The configuration of the devices in system 200 of FIG. 2 is merely one physical system on which the disclosed embodiments can be executed. Other configurations of the devices shown can exist to practice the disclosed embodiments. Further, configurations of additional or fewer devices than the devices shown in FIG. 2 can exist to practice the disclosed embodiments. Additionally, the devices shown in FIG. 2 can be combined to allow for fewer devices than shown or can be separated such that more than the three devices exist in a system. It will be appreciated that many various combinations of computing devices can execute the methods and systems disclosed herein. Examples of such computing devices can include other types of medical devices and sensors, infrared cameras/detectors, sensors that detect other portions of the electromagnetic spectrum, night vision cameras/detectors, other types of cameras, augmented reality goggles, virtual reality goggles, mixed reality goggle, radio frequency transmitters/receivers, smart phones, personal computers, servers, laptop computers, tablets, blackberries, RFID enabled devices, smart watch or wearables, or any combinations of such devices.

Referring back to FIG. 1, the display 122 can be used to display various information regarding the patient 112 monitored by the system 100. In some embodiments, the system 100, including the camera 114, the computing device 115, and the hardware processor 118, is used to generate sensor data (e.g., captured images and/or generated distance signals) and, by a signal processor, determine classifications of patient motion that can be displayed in a user interface presented on the display 122 or otherwise indicated as described with respect to system 200 of FIG. 2. Additionally or alternatively, the system 100, including the camera 114, the computing device 115, and the hardware processor 118, is used to generate, receive, and/or display respiratory measurement data (e.g., respiratory rate) in the same or a different user interface. Additionally or alternatively, the system 100, including the camera 114, the computing device 115, and the hardware processor 118, is used to generate, receive, and/or display the generated sensor data as an image of the patient and/or the ROI.

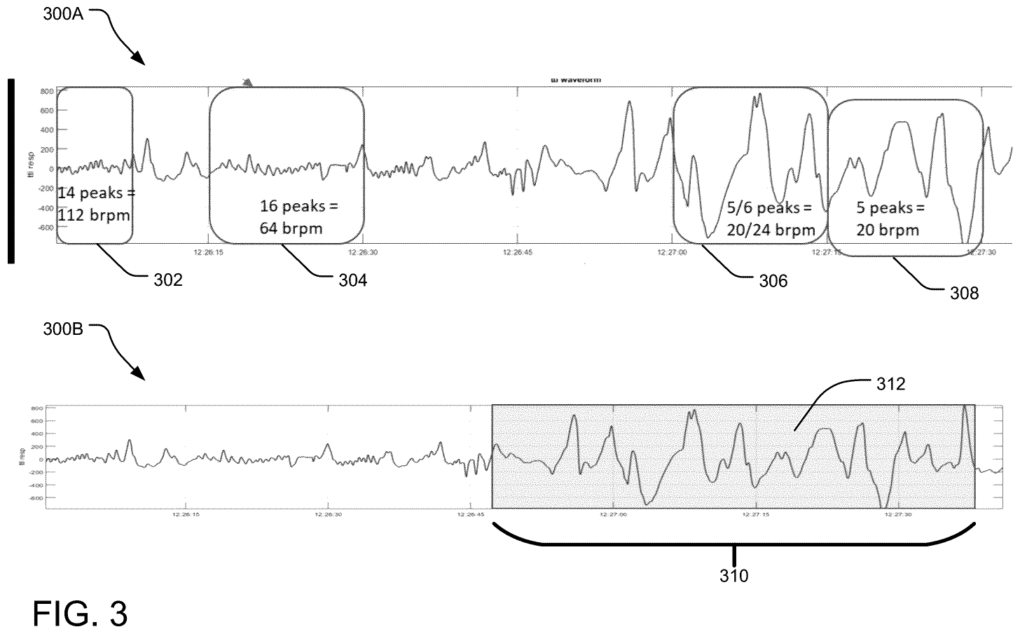

FIG. 3 is a plot view of line plots illustrating signals from a transthoracic impedance respiratory measurement as a function of time. The transthoracic impedance respiratory measurement indicates a magnitude of signal representing current passed through a chest of the patient. The transthoracic impedance measurement is only an example respiratory measurement method illustrated to demonstrate the varying veracity of a respiratory measurement during durations in which patient motion is attributable to one or more of respiratory patient motion and non-respiratory patient motion. A transthoracic impedance (TTI) device introduces a current to a patient and measures the output response current to determine an impedance measurement. Impedance measurements vary during respiration, indicating the alternating behavior of inhaling and exhaling. Although plots 300A and 300B are derived from TTI measurements, implementations of the technology described herein are contemplated in which respiratory measurements such as raw respiratory motion signals and determined respiratory rates may be derived by other methods, such as captured image analysis and/or depth or distance signal analysis as disclosed herein.

In the illustrated implementation, a first plot 300A plots transthoracic impedance (TTI) signals over a duration (e.g., over a predefined time or number of samples). Over a first duration 302, it can be seen that the peaks and troughs of the signal are relatively similar in magnitude and display relatively regular oscillation. The first duration 302 may be indicative of respiratory patient motion substantially devoid of non-respiratory patient motion. Counting the peaks, the first duration 302 is indicative of a respiratory rate of about 112 breaths per minute. Over a second duration 304, the signal begins similarly to the first duration 302 signal, but larger peaks with less clear oscillatory behavior appear towards the end of the second duration 304. These large peaks with less oscillatory behavior are indicative of gross, non-respiratory patient motions. The large peaks representing the gross motion obfuscate or drown out the smaller and more regular oscillatory signals indicative of respiratory patient motion. Because the gross movement obscures the oscillatory nature of the respiratory patient motion signals towards the end of the second duration 304, the respiratory rate derived from the TTI will indicate when counting the peaks that the respiration rate has dropped to about 64 breaths per minute. If the patient motion did not include non-respiratory patient motion, this drop in respiratory rate would indicate a significant decline in the health of the patient.

Further still, looking to a third duration 306 and a fourth duration 308, the TTI signal during the entire durations 306, 308 is dominated by responses indicative of non-respiratory patient motion. The third and fourth durations 306, 308 indicate respiratory rates of between 20 and 24 breaths per minute, representing a considerable decline in respiration if the system does not account for the non-respiratory patient motion to which the errant respiratory measurements can be attributed. A second plot 300B is illustrated as an exemplary element that could be presented in a user interface. The plot includes an overlaid indication of veracity displayed as a highlighted area 312 of the second plot 300B overlaid over the illustrated TTI signal. The highlighted area 312 highlights the second plot 300B over a portion representing a fifth duration 310, during which the magnitudes of peaks and oscillatory behavior indicate that the TTI signal is dominated by measurements associated with gross, non-respiratory patient motion. The highlighting may indicate to a health professional or caretaker that the TTI signals and respiratory rates derived therefrom have limited veracity and should potentially be ignored.

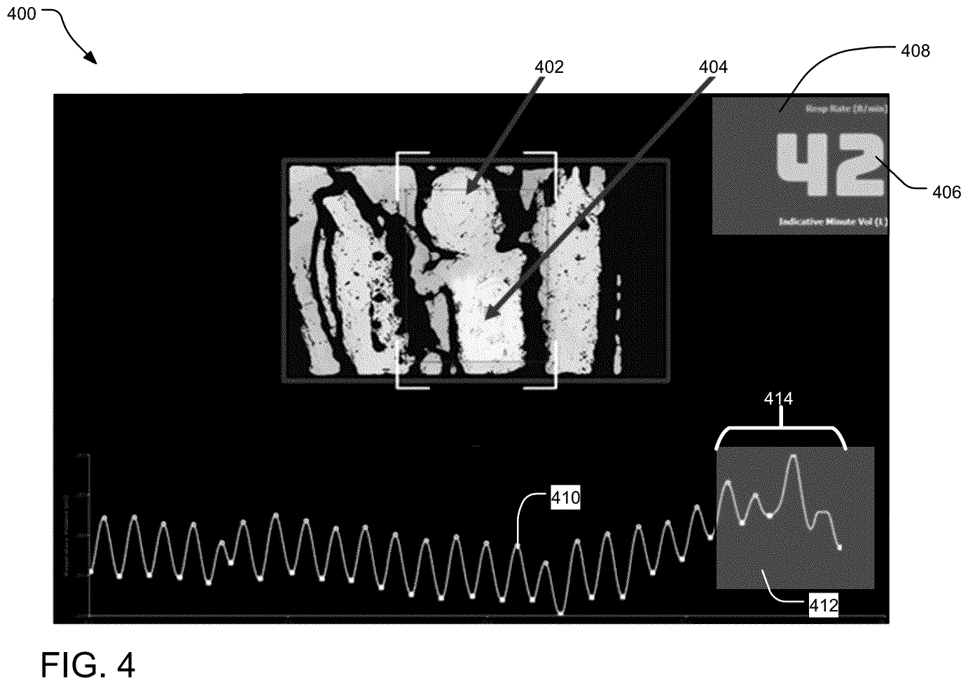

FIG. 4 is a display view for an implementation of a user interface 400 of a video-based patient monitoring system configured in accordance with various embodiments of the present technology. In implementations, the user interface 400 may include one or more of a visual representation of a patient 402, an ROI 404 of the patient, a respiratory rate 406 of the patient, an indication of the veracity of the respiratory rate 406, a respiratory measurement signal 410, and an indication of the veracity of the respiratory measurement signal 412 over a duration 414. In the illustrated implementation, the user interface 400 includes the visual representation of the patient 402 and the ROI 404. In the illustrated user interface 400, the ROI 404 is highlighted to indicate the portion of the patient that represents the ROI 404. In implementations, the system may modify the visual representation of the ROI 404 to indicate one or more of the veracity of respiratory measurements, a degree of lethargy of the patient, and the like. The illustrated user interface 400 further includes the indication of a respiratory rate 406 and the indication of veracity 408 of the respiratory rate 406 by an overlaid highlighting of the respiratory rate 406. The illustrated user interface 400 further includes the respiratory measurement signal 410, depicting magnitudes of a signal representing respiratory behavior over time. The indication of the veracity of the respiratory measurement signal 412 is presented relative to the duration 414 over which non-respiratory patient motion is detected and classified by the system. In implementations, the respiratory rate 406 is derived from the respiratory measurement signal 410.

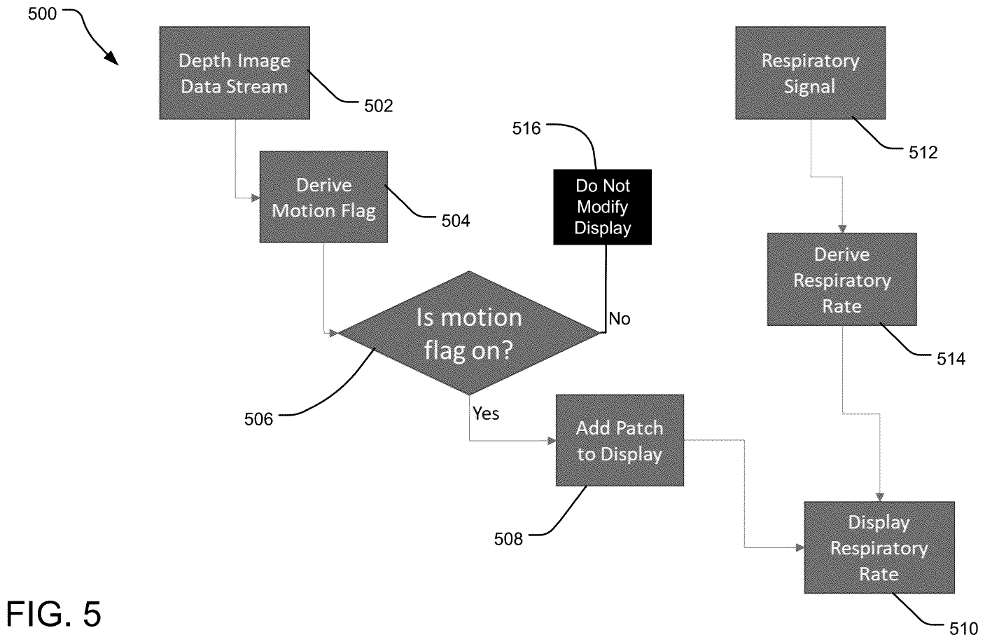

FIG. 5 is a flow chart of an implementation of a method 500 for providing an informative display of data obtained from non-contact monitoring of a patient configured in accordance with various embodiments of the present technology. In step 502, a depth image data stream is generated, which includes data representing one or more of images captured of an ROI or distances detected between the ROI and a distance sensor (e.g., of a depth-sensing camera).

In step 504, a motion classification flag is derived. In the illustrated implementation, a motion flag represents an instruction for displaying the veracity of respiratory measurement. The instruction, including the motion classification flag, may be generated by a signal processor.

The signal processor may process generated sensor data generated by an image capture device. The signal processor receives the generated sensor data and is operable to determine whether the generated sensor data satisfies a non-respiratory motion condition. The non-respiratory motion condition and/or its satisfaction may be determined based on a predefined relationship accessible to the signal processor between generated sensor data and the motion of a patient attributable to patient actions other than respiration. Non-respiratory patient motions attributable to patient actions other than respiration can affect the veracity of respiratory measurements (e.g., respiratory rate) by obfuscating signals associated with respiratory patient motions that are typically smaller in magnitude and show regular oscillations associated with alternating lung expansion and relaxation.

The non-respiratory motion condition may include a range or threshold value of relevant metrics such as changes in distances, asymmetrical changes of distances (e.g., relative to a medial ROI axis), changes in pixel values in capture images, and the like. In an implementation, asymmetric motion detected in an ROI on a patient's chest can indicate the motion is attributable to something other than breathing. In another implementation, a magnitude of motion in the ROI may be indicative of a gross patient movement not associated with patient breathing.

The non-respiratory condition may be based on a demographic of the patient. Patient demographics upon which the non-respiratory condition may be based can include one or more of age, size, race, gender, body type, and the like. For example, a specific non-respiratory condition may apply to neonates. Neonates may present very significant differences between respiratory neonate patient motion and non-respiratory neonate patient motion than adults. Also, non-respiratory neonate patient motion may be more uniform among neonates than non-respiratory adult patient motion is among adults.

The signal processor may include a patient motion classifier operable to classify patient motion based on the satisfaction of one or more non-respiratory motion conditions. For example, a single patient motion classification can be based on satisfaction of more than one non-respiratory condition. Because the classifications represent an extent to which patient motion is attributable to non-respiratory patient motion, and non-respiratory motion is correlated with poor veracity in respiratory measurements, the classifications of the patient motion may represent the veracity of respiratory measurements (e.g., based on predefined parameters of veracity).

In implementations, the signal processor may be operable to determine a level of lethargy of a patient based on the generated sensor data. For example, in neonates, the baseline activity of the neonate reflected in non-respiratory movements can be an indicator of the health of a neonate. The signal processor may determine the lethargy using a predefined relationship between the generated sensor data generated over a first duration relative to the generated sensor data generated over a second duration, or the generated sensor data may be compared with preexisting data associated with lethargy to the satisfaction of a lethargy condition (e.g., a threshold relative motion pattern predefined to indicated lethargy). Levels of lethargy may be elements of predefined relationships between generated sensor data and non-respiratory patient motion and/or may be elements of non-respiratory motion conditions associated with the predefined relationships.

In implementations, the patient motion may be classified in a binary fashion as one of respiratory patient motion or of non-respiratory patient motion (e.g., sufficient gross non-respiratory patient motion to flag measurements made during the time of the motion as lacking a predefined requisite veracity). In other implementations, the patient motion may be classified based on an extent of motion attributable to each of respiratory patient motion and non-respiratory patient and may additionally or alternatively include or be based on a determined confidence that the patient motion is attributable to respiratory motion and/or non-respiratory motion. The motion classifications can be representative of different degrees of the patient motion's attributability to non-respiratory patient motion and/or different degrees of confidence in the determination of the attributability. For example, the motion classifier may generate one or more of a score associated with the determination of the attributability and a score associated with confidence in the determination of the attributability to classify patient motion. In implementations in which a confidence score is determined, the confidence score may be combined with the score associated with the attributability (e.g., multiply or otherwise weigh) to classify patient motion.

In implementations, the patient motion classifier associates each motion classification with a derived motion classification flag and derives the motion classification flag when the motion classifier classifies an associated patient motion. The motion classification flag may be set to inform elements of the system of a classification of patient motion.

The signal processor can generate instructions for the display of data generated by the signal processor. An instruction can include data representing an instruction to display an indication of veracity of a respiratory measurement generated during a duration of analysis of patient motion based on the classification of the patient motion. The instruction may alternatively or additionally include data representing a respiratory measurement (e.g., one generated by the signal processor). In implementations, the instruction and/or indication may include data representing one or more of a motion classification flag, a classification-specific display, an overlaid display (e.g., configured to overlay a displayed element in a user interface), an image representation of patient motion (e.g., a visual or video representation of captured images and/or generated distance signals), an audio signal, a flashing element (e.g., where the magnitude of light of elements of a display is alternatively increased and decreased), a different alert, a code representing the aforementioned items, and the like. In implementations in which the classification is one of a number of classifications, each classification may correspond to a different display. The signal processor may output data representing the motion classification or a specific display associated in memory of the system 200 with the motion classification. For example, different classifications can be represented in a display by different colors, different magnitudes of light in the display, different frequencies at which to flash light in the display. In implementations in which the motion classifications are representative of different degrees of the patient motion's attributability to non-respiratory patient motion and/or different degrees of confidence in the determination of the attributability, the corresponding display may represent a spectrum or range of color, light magnitude, or flash frequency based on a magnitude of the one or more of the different degrees of the patient motion's attributability to non-respiratory patient motion and/or different degrees of confidence in the determination of the attributability. In the illustrated implementation, the signal processor outputs an instruction including a derived motion classification flag.

Step 506 is a decision block that determines whether the motion classification flag is on. If the instruction output by the signal processor includes the derived motion flag, then the method proceeds to step 508, in which the system instructs that a patch (e.g., an overlaid indication or other indication of non-respiratory motion) be added to a displayed user interface. If the instruction output by the signal processor does not include the derived motion flag (or if no instruction is generated in implementations in which instructions are generated only when non-respiratory motion is detected), then the display is not modified in step 516.

In step 512, a respiratory signal is determined representative of magnitudes of respiratory patient motion, and in step 514, a respiratory rate (or another respiratory measurement) is derived from the determined respiratory signal. In implementations, the signal processor can determine other respiratory measurements such as values of a variety of parameters (e.g., respiratory patient motion, non-respiratory patient motion, tidal volume, minute volume, respiratory rate, etc.) based on a respiratory signal (e.g., generated sensor data). In implementations, the signal processor can determine the respiratory measurements based on the generated sensor data. In other implementations, the signal processor can receive respiratory measurements generated by other means, including one or more of a transthoracic impedance measurement, an electrocardiogram, capnograph, spirometer, pulse oximeter, and a manual user entry. Implementations are also contemplated in which the signal processor does not process respiratory measurements other than the classification of patient motion.

In implementations, the respiratory measurements may inform when the signal processor classifies patient motion. For example, the signal processor may be configured to monitor patient motion and/or generate sensor data responsively to the detection of anomalous respiratory measurements (e.g., when the monitored respiratory measurements satisfy an anomalous respiratory measurement condition based on a predefined threshold or range of values of the respiratory measurements or changes in the respiratory measurements). In implementations, determinations and/or outputs of the signal processor can be sent to systems storing electronic records to add the outputs to the electronic records. In an alternative implementation, the signal processor is not involved in the generation of the respiratory signal in step 512 or the derivation of the respiratory rate therefrom in step 514. For example, a display system for displaying the respiratory rate (or another respiratory measurement) may be communicatively coupled to a different system for conducting steps 512 and/or 514.

In step 510, a respiratory rate (and/or another respiratory measurement) is displayed in a user interface of a display. If the motion classification flag is determined to be on in step 506 and the patch (or other indication of the veracity of a respiratory measurement) is consequently instructed to be added to a user interface of the display. While illustrated as a displayed item, an indication of the veracity of the measured respiratory rate (or another respiratory measurement) can be any alert presented to a user (e.g., as a visual display or audible alert).

In implementations, the instruction or indication, including the patch or other indication of the veracity of the respiratory measurement, may be configured to cause the display to display the indication of the classification as an element overlaid over or underlaid under (e.g., displayed behind) another displayed respiratory measurement or an image of the patient including an ROI image to indicate the veracity of the displayed respiratory measurement. For example, the display may be configured to display a measured respiratory rate and may display the indication of the veracity of the measured respiratory rate over or under the displayed respiratory rate. An overlaid or underlaid display of the indication may be configured to be visually contrasted from the displayed respiratory measurement. For example, the elements may be of different colors and/or may be of different transparencies. For example, the displayed indication of veracity may be at least partially transparent to maintain visibility of the displayed respiratory measurement (e.g., appearing as a highlighting of or patch over the displayed respiratory measurement). Additionally or alternatively, the displayed indication of veracity may be overlaid over an ROI image. In an alternative implementation, the instruction instructs a display to not display the respiratory measurements when patient motion is classified as non-respiratory motion and/or display that the non-respiratory motion is occurring. Other implementations are contemplated in which the indication of the veracity of the respiratory measurement is displayed in a separate portion from but concurrently with the respirator measurement.

In other implementations, the user interface may determine to not display the respiratory measurements during durations when the signal processor determines that the respiratory measurement is of low veracity (e.g., when a patient motion is classified as non-respiratory patient motion) and/or the user interface may display the indication of the veracity of the respiratory measurement instead of the respiratory measurement.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. Furthermore, the various embodiments described herein can also be combined to provide further embodiments.

The systems and methods described herein can be provided in the form of tangible and non-transitory machine-readable medium or media (such as a hard disk drive, hardware memory, etc.) having instructions recorded thereon for execution by a processor or computer. The set of instructions can include various commands that instruct the computer or processor to perform specific operations such as the methods and processes of the various embodiments described here. The set of instructions can be in the form of a software program or application. The computer storage media can include volatile and non-volatile media, and removable and non-removable media, for storage of information such as computer-readable instructions, data structures, program modules, or other data. The computer storage media can include but are not limited to RAM, ROM, EPROM, EEPROM, flash memory or other solid-state memory technology, CD-ROM, DVD, or other optical storage, magnetic disk storage, or any other hardware medium which can be used to store desired information and that can be accessed by components of the system. Components of the system can communicate with each other via wired or wireless communication. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent that any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded. Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may, in some cases, depend on the specific context. However, generally speaking, the nearness of completion will be so as to have the same overall result as if absolute and total completion were obtained. The use of “substantially” is equally applicable when used in a negative connotation to refer to the complete or near-complete lack of an action, characteristic, property, state, structure, item, or result. As used herein, terms such as “substantially,” “about,” “approximately,” or other terms of relative degree are interpreted as a person skilled in the art would interpret the terms and/or amount to a magnitude of variability of one or more of 1%, 2%, 3%, 4%, 5%, 6%, 7%, 8%, 9%, 10%, 11%, 12%, 13%, 14%, or 15% of a metric relative to the quantitative or qualitative feature described. For example, a term of relative degree of “about a first time” suggests the timing may have a magnitude of variability relative to the first time. When values are presented herein for particular features and/or a magnitude of variability, ranges above, ranges below, and ranges between the values are contemplated.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structures or any other physical structure suitable for implementation of the described techniques. Also, the techniques could be fully implemented in one or more circuits or logic elements.

I/We claim:

1. An image-based patient monitoring method, comprising:
capturing a first image, by an image capture sensor, of a region of a patient at a first time;
capturing a second image, by the image capture sensor, of the region of the patient at a second time;
determining whether the a comparison between the first captured image and the second captured image satisfies a non-respiratory motion condition, the non-respiratory motion condition based in part on a relationship between captured images and motion of a patient attributable to patient actions other than respiration of the patient;
classifying a motion of the patient as non-respiratory motion when the non-respiratory motion condition is satisfied; and
transmitting an instruction to display on a graph plotting the patient's respiratory measurements taken over time a visual indicator between the first time and the second time, the visual indicator providing an indication of whether the motion of the patient has been classified as non-respiratory motion.

2. The method of claim 1, further comprising:
generating a first distance signal based on at least one first distance between at least one point in the region of the patient and a distance sensor of the image capture sensor at about the first time; and
generating a second distance signal based on at least one second distance from the at least one point in the region of the patient and the distance sensor at about the second time,
wherein the determining is further based on the first generated distance signal and the second generated distance signal, and the relationship is further between generated distance signals, the captured images, and the motion of a patient.

3. The method of claim 2, wherein the non-respiratory motion condition includes a magnitude of motion threshold based on one or more of the first captured image, the second captured image, the first generated distance signal, and the second generated distance signal.

4. The method of claim 2, wherein the non-respiratory motion condition includes whether the first generated distance signal and the second generated distance signal represent asymmetric movement of portions of the predefined region relative to a medial axis of the region to satisfy an asymmetric motion condition.

5. The method of claim 1, wherein the patient's respiratory measurement taken over time are obtained from one or more of a transthoracic impedance measurement, an electrocardiogram, capnograph, spirometer, pulse oximeter, and a manual user entry.

6. The method of claim 1, wherein the visual indicator comprises an overlay on the graph plotting the patient's respiratory measurements taken over time.

7. The method of claim 1, further comprising:
analyzing respiratory activity of the patient based on respiratory measurements determined exclusively during periods in which the motion of the patient is classified as attributed to respiration of the patient.

8. An image-based patient monitoring system, comprising:
one or more hardware processors operable to execute instructions stored in memory;
a distance sensor operable to:
generate a first distance signal based on a detected at least one first distance between at least one point in a region of a patient and the distance sensor at a first time; and
generate a second distance signal based on a detected at least one second distance from the at least one point in the region of the patient and the distance sensor at a second time;
a signal processor executable by the one or more hardware processors and operable to:
determine whether the first generated distance signal and the second generated distance signal satisfy a non-respiratory motion condition, the non-respiratory motion condition based in part on a relationship between generated distance signals and motion of a patient attributable to patient actions other than respiration of the patient; and
classify a motion of the patient as non-respiratory motion when the non-respiratory motion condition is satisfied; and
an instruction generator executable by the one or more hardware processors and operable to generate an instruction to display on a graph plotting the patient's respiratory measurements taken over time a visual indicator between the first time and the second time, the visual indicator providing an indication of whether the motion of the patient has been classified as non-respiratory motion.

9. The image-based patient monitoring system of claim 8, further comprising:
an image capture sensor operable to:
capture a first image of the region of a patient at about the first time; and
capture a second image of the region of the patient at about the second time,
wherein the signal processor is operable to determine whether the first generated distance signal and the second generated distance signal satisfy a non-respiratory motion condition further based on the first captured image and the second captured image, and the relationship is further between captured images, the generated distance signals, and the motion of a patient.

10. The image-based patient monitoring system of claim 9, wherein the non-respiratory motion condition includes a magnitude of motion threshold based on one or more of the first captured image, the second captured image, the first generated distance signal, and the second generated distance signal.

11. The image-based patient monitoring system of claim 9, wherein the non-respiratory motion condition includes that the first generated distance signal and the second generated distance signal represent asymmetric movement of the region relative to a medial axis of the region to satisfy an asymmetric motion condition.

12. The image-based patient monitoring system of claim 9, wherein the patient's respiratory measurement taken over time are obtained from one or more of a transthoracic impedance measurement, an electrocardiogram, capnograph, spirometer, pulse oximeter, and a manual user entry.

13. The image-based patient monitoring system of claim 9, wherein the visual indicator comprises an overlay on the graph plotting the patient's respiratory measurements taken over time.

14. The image-based patient monitoring system of claim 9, further comprising:
a respiratory activity analyzer to analyze respiratory activity of the patient based on respiratory measurement data determined exclusively during periods in which the motion of the patient is classified as attributed substantially to respiration of the patient.
